(19) United States
(12) Patent Application Publication
(10) Pub. No.: US 2007/0086960 A1
(43) Pub. Date: Apr. 19, 2007

(54) Systems and methods for enhancing the appearance of teeth

(76) Inventors: J. Gail Tarver, Lehi, UT (US); Ben D. Card, Mapleton, UT (US); Michael R. LaDow, Provo, UT (US); Donald H. Livingstone, Provo, UT (US)

Correspondence Address: KIRTON AND MCCONKIE, 60 EAST SOUTH TEMPLE,, SUITE 1800, SALT LAKE CITY, UT, 84111, US

(21) Appl. No.: 11/514,680

(22) Filed: Sep. 1, 2006

Related U.S. Application Data
(63) Continuation-in-part of application No. 11/217,275, filed on Sep. 1, 2005.
Application No. 11/217,275 is a continuation-in-part of application No. 10/442,559, filed on May 21, 2003.

(51) Int. Cl. A61K 31/42

(52) U.S. Cl. 424/49

(57) ABSTRACT

Effective, non-invasive and non-destructive systems and methods provide an appearance of increased whiteness and/or brightness of the teeth, thereby enhancing the appearance of the teeth. One or more residual colors or dyes are applied to the surface of a tooth in order to complete the color spectrum of the tooth surface or more closely complete the color spectrum of the tooth surface, thereby enhancing the appearance of the tooth since a complete color spectrum appears as white. In one implementation, a residual color includes a shade of violet, wherein a red-violet reduces the yellow appearance of the tooth and a blue-violet enhances the brightness of the tooth. In one implementation, a bleaching step is performed to the surface of the tooth followed by the application of a residual color onto the surface of the tooth. In one implementation, a residual color includes a yellow shade applied to the surface of a tooth that otherwise appears gray to cause the tooth to appear whiter.

### RELATED APPLICATIONS

This application is a continuation-in-part of U.S. patent application Ser. No. 11/217,275 filed Sep. 1, 2005 entitled TOOTH WHITENING COMPOSITIONS AND METHODS FOR USING THE SAME, which is a continuation-in-part of U.S. patent application Ser. No. 10/442,559 filed May 21, 2003 entitled TOOTH WHITENING COMPOSITIONS AND METHODS FOR USING THE SAME, which are both incorporated herein by reference.

### BACKGROUND OF THE INVENTION

1. Field of the Invention

The present invention relates to enhancing the appearance of teeth. In particular, the present invention relates to effective, non-invasive and non-destructive systems and methods that provide an appearance of increased whiteness and/or brightness of the teeth, thereby enhancing the appearance of the teeth.

2. Background and Related Art

Tooth bleaching has become popular as people realize the cosmetic benefits of having whiter teeth. As a result, some tooth bleaching techniques are available for use in a dentist's office (e.g., laser bleaching) while other tooth bleaching techniques include home bleaching kits that can either be purchased from a dentist or via mail order television advertisements. Some tooth bleaching techniques utilize compositions having relatively low concentrations of bleaching agents and may take weeks or even months to complete. Other tooth bleaching techniques utilize more concentrated and powerful bleaching compositions used in dental offices in order to show more immediate results.

Although current tooth bleaching techniques have found widespread acceptance, the techniques are not without problems. For example, presently known techniques used to whiten teeth rely on invasive chemical components to break down pigments internally disposed in teeth. Such chemical components can cause sensitivity in teeth, sensitivity in gingival tissues, and/or pain. As a result of such tooth sensitivity, desensitizing agents have been used to reduce the tooth sensitivity. However, such desensitizing agents have not eliminated or reduced the root cause of sensitivity, which is the use of bleaching agents, such as peroxide-based bleaching agents, as the sole means for whitening. If anything, the use of desensitizing agents has allowed for the use of even harsher and more concentrated bleaching compositions.

Another problem relates to the fact that the bleaching compositions, the associated equipment and/or the dentist labor costs can be relatively high. Thus, less potent compositions are available for home use as over-the-counter products. Although such compositions avoid the disadvantages of more potent bleaching systems, such over-the-counter compositions intended for home use are typically less effective in removing or concealing tooth discoloration and typically require increased frequency and duration of use compared to professionally applied compounds.

Accordingly, while techniques currently exist that are used to bleach teeth, challenges still exist. Accordingly, it would be an improvement in the art to augment or even replace current techniques with other techniques.

### SUMMARY OF THE INVENTION

The present invention relates to enhancing the appearance of teeth. In particular, the present invention relates to effective, non-invasive and non-destructive systems and methods that provide an appearance of increased whiteness and/or brightness of the teeth, thereby enhancing the appearance of the teeth.

Implementation of the present invention takes place in association with applying one or more residual colors or dyes to the surface of a tooth in order to complete the color spectrum of the tooth surface or more closely complete the color spectrum of the tooth surface, thereby enhancing the appearance of the tooth since a complete color spectrum appears as white.

In some implementations, one or more residual colors are applied to the surface of the tooth to enhance the appearance thereof. In one implementation, a residual color includes a shade of violet, wherein a red-violet reduces the yellow appearance of the tooth and a blue-violet enhances the brightness of the tooth.

In some implementations, a two step process is utilized wherein a red-violet residual color is applied to the surface of a tooth in a first step to enhance the color of the tooth, causing the tooth to appear whiter. In a second step, a blue-violet residual color is applied to the surface of the tooth to enhance the brightness of the tooth, causing the tooth to appear brighter. In another implementation, the blue-violet residual color is applied first and then the red-violet residual color is applied second. In yet another implementation, the blue-violet residual color and the red-violet residual color are simultaneously applied.

In a further implementation, the one or more residual colors are applied through utilization of a carrier. Examples of carriers include a gel (e.g., a glycerin or other type of gel), a toothpaste, a mouth wash, a liquid or beverage, a solid, a gas, a food, or any other type of carrier that is capable of applying a dye to the surface of a tooth.

In one implementation, a bleaching step is performed to the surface of the tooth followed by the application of a residual color onto the surface of the tooth. In a further embodiment, the application of the residual color in combination with a step of bleaching the surface of the tooth effectively tones the tooth and thus enhances the appearance of the tooth. Further, application of the residual color prevents over bleaching of the tooth surface.

In some implementations, the residual color comprises a dual dye. In other implementations, the residual color comprises a single dye.

In some implementations, an intensity range is utilized based upon the current color of the tooth surface. Thus, for example, a more intense residual color is applied to a darker tooth surface color to enhance the appearance of the tooth. Accordingly, the intensity of the residual color is directly proportional to the darkness of the tooth prior to application of the residual color, whether the darkness of the tooth is due to natural colors and/or stains to the surface of the tooth. While implementations embrace all types of intensities, some implementations embrace an intensity of the dye or residual color being no more than 1% of the total volume or weight of the product being applied to the tooth surface. In other implementations, the intensity of the dye or residual color is no more than 5% of the total volume or weight of the product being applied to the tooth surface. In other implementations, the intensity of the dye or residual color is no more than 10% of the total volume or weight of the product being applied to the tooth surface. In other implementations, the intensity of the dye or residual color is more than 10% of the total volume or weight of the product being applied to the tooth surface.

In a further implementation, a residual color includes a yellow shade applied to the surface of a tooth that appears gray. The application of a residual color that includes a yellow shade enhances the appearance of the tooth surface, by causing the tooth to appear whiter.

In some implementations, a tooth whitening system is provided that effectively conceals tooth discoloration within a short period of time. In one implementation, a tooth whitening system is provided that incorporates a dye having a dominant peak absorption wavelength to effectively mask tooth discoloration. In another implementation, a tooth whitening system is provided that is capable of topical application that incorporates a spectrally pure dye to effectively mask tooth discoloration.

In another implementation, a tooth whitening composition is provided that comprises a food product capable of being deposited onto a person's teeth during ordinary consumption. In this manner, the tooth whitening composition of the present invention minimizes the time and effort required for tooth whitening while maximizing whitening results.

In one implementation, a topically applied tooth whitening system is provided to enhance the whitening effect without causing tooth sensitivity. In one implementation, an inexpensive tooth whitening system is provided that is safe for home use. In one implementation of the present invention, a tooth whitening system is provided that is capable of producing effective results within a short period of time after its initial application.

While the methods and processes of the present invention have proven to be particularly useful in the area of enhancing teeth by making them appear white, those skilled in the art can appreciate that the methods and processes can be used in a variety of different applications to enhance the appearance of teeth in a variety of different manners, including other characteristics, including brightness.

These and other features and advantages of the present invention will be set forth or will become more fully apparent in the description that follows and in the appended claims. The features and advantages may be realized and obtained by means of the instruments and combinations particularly pointed out in the appended claims. Furthermore, the features and advantages of the invention may be learned by the practice of the invention or will be obvious from the description, as set forth hereinafter.

### BRIEF DESCRIPTION OF THE DRAWINGS

In order that the manner in which the above recited and other features and advantages of the present invention are obtained, a more particular description of the invention will be rendered by reference to specific embodiments thereof, which are illustrated in the appended drawings. Understanding that the drawings depict only typical embodiments of the present invention and are not, therefore, to be considered as limiting the scope of the invention, the present invention will be described and explained with additional specificity and detail through the use of the accompanying drawings in which:

FIG. 1 illustrates a comparative chart of ranges of wavelengths, colors absorbed, and corresponding reflected or otherwise visually apparent colors;

FIG. 2 illustrates a representative spectral analysis showing a comparison between the absorption spectra of dyes having peak absorption wavelengths in the visible range of red and blue, compared to a dye having a dominant peak absorption wavelength in the visible range of violet;

FIG. 3 illustrates a spectral analysis of a dye in accordance with a representative embodiment of the present invention;

FIG. 4 illustrates a spectral analysis of a dye in accordance with another embodiment of the present invention;

FIG. 5 illustrates a spectral analysis of a dye in accordance with another embodiment of the present invention; and

FIG. 6 illustrates a spectral analysis of a dye in accordance with another embodiment of the present invention.

### DETAILED DESCRIPTION OF THE INVENTION

The present invention relates to enhancing the appearance of teeth. In particular, the present invention relates to effective, non-invasive and non-destructive systems and methods that provide an appearance of increased whiteness and/or brightness of the teeth, thereby enhancing the appearance of the teeth.

At least some embodiments of the present invention take place in association with applying one or more residual colors or dyes to the surface of a tooth in order to complete the color spectrum of the tooth surface or more closely complete the color spectrum of the tooth surface, thereby enhancing the appearance of the tooth since a complete color spectrum appears as white.

In some embodiments, one or more residual colors are applied to the surface of the tooth to enhance the appearance thereof. In one embodiment, a residual color includes a shade of violet, wherein a red-violet reduces the yellow appearance of the tooth and a blue-violet enhances the brightness of the tooth.

In some embodiments, a two step process is utilized wherein a red-violet residual color is applied to the surface of a tooth in a first step to enhance the color of the tooth, causing the tooth to appear whiter. In a second step, a blue-violet residual color is applied to the surface of the tooth to enhance the brightness of the tooth, causing the tooth to appear brighter. In another embodiment, the blue-violet residual color is applied first and then the red-violet residual color is applied second. In yet another embodiment, the blue-violet residual color and the red-violet residual color are simultaneously applied.

In a further embodiment, the one or more residual colors are applied through utilization of a carrier. Examples of carriers include a gel (e.g., a glycerin or other type of gel), a toothpaste, a mouth wash, a liquid or beverage, a solid, a gas, a food, or any other type of carrier that is capable of applying a dye to the surface of a tooth.

In one embodiment, a bleaching step is performed to the surface of the tooth followed by the application of a residual color onto the surface of the tooth. In a further embodiment, the application of the residual color in combination with a step of bleaching the surface of the tooth effectively tones the tooth and thus enhances the appearance of the tooth. Further, application of the residual color prevents over bleaching the tooth surface.

In some embodiments, the residual color comprises a dual dye. In other embodiments, the residual color comprises a single dye.

In some embodiments, an intensity range is utilized based upon the current color of the tooth surface. Thus, for example, a more intense residual color is applied to a darker tooth surface color to enhance the appearance of the tooth. Accordingly, the intensity of the residual color is directly proportional to the darkness of the tooth prior to application of the residual color, whether the darkness of the tooth is due to natural colors and/or stains to the surface of the tooth. While embodiments embrace all types of intensities, some implementations embrace an intensity of the dye or residual color being no more than 1% of the total volume or weight of the product being applied to the tooth surface. In other embodiments, the intensity of the dye or residual color is no more than 5% of the total volume or weight of the product being applied to the tooth surface. In other embodiments, the intensity of the dye or residual color is no more than 10% of the total volume or weight of the product being applied to the tooth surface. In other embodiments, the intensity of the dye or residual color is more than 10% of the total volume or weight of the product being applied to the tooth surface.

In a further embodiment, a residual color includes a yellow shade applied to the surface of a tooth that appears gray. The application of a residual color that includes a yellow shade enhances the appearance of the tooth surface, by causing the tooth to appear whiter.

In some embodiments, a tooth whitening system is provided that effectively conceals tooth discoloration within a short period of time. In one embodiment, a tooth whitening system is provided that incorporates a dye having a dominant peak absorption wavelength to effectively mask tooth discoloration. In another embodiment, a tooth whitening system is provided that is capable of topical application that incorporates a spectrally pure dye to effectively mask tooth discoloration.

In another embodiment, a tooth whitening composition is provided that comprises a food product capable of being deposited onto a person's teeth during ordinary consumption. In this manner, the tooth whitening composition of the present invention minimizes the time and effort required for tooth whitening while maximizing whitening results.

In one embodiment, a topically applied tooth whitening system is provided to enhance the whitening effect without causing tooth sensitivity. In one embodiment, an inexpensive tooth whitening system is provided that is safe for home use. In one embodiment of the present invention, a tooth whitening system is provided that is capable of producing effective results within a short period of time after its initial application.

While the methods and processes of the present invention have proven to be particularly useful in the area of enhancing teeth by making them appear white, those skilled in the art can appreciate that the methods and processes can be used in a variety of different applications to enhance the appearance of teeth in a variety of different manners, including other characteristics, including brightness.

Embodiments of the present invention may be embodied in various forms without departing from its spirit or essential characteristics. The embodiments described herein are thus to be considered in all respects only as illustrative and not restrictive. The scope of the invention is, therefore, indicated by the appended claims rather than by the following descriptions. All changes that come within the meaning and range of equivalency of the claims are to be embraced within their scope.

The term “dye” can refer to any FD&C dye, D&C dye, FD&C lake, acid dye, or natural food color such as carmine, carotene, annatto, turmeric, curcumin, blueberry, or other natural food colors known in the art, whether used individually or in combination, or any other dye that can be used to apply a residual color to the surface of a tooth. The term “food product” can refer to any nutriment in solid, liquid or gaseous form that is safe either for human consumption or for application to the mucous membranes of the mouth (including mouthwashes, dentifrices, and the like).

As used in this specification, “visible spectrum” and “light” can refer to the band of electromagnetic radiation ranging from wavelengths of approximately 400 to approximately 700 nm, corresponding to the sensitivity of the human eye.

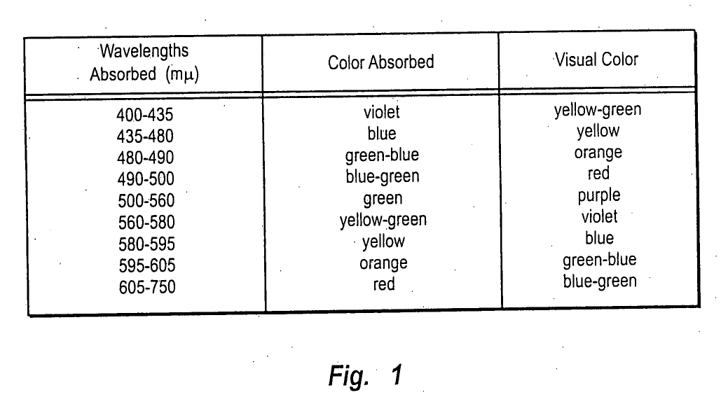

The human eye has three types of cone cells that have peak responses to light with wavelengths that correspond to red, green, and blue-violet (the wavelengths corresponding to these colors being set forth in FIG. 1). “White light” refers to any electromagnetic radiation that stimulates all three types of cone cells in the human eye—red, green, and blue-violet—in substantially the same proportions as do the emissions from a body at a white heat. We can designate the stimulation of the three types of cone cells by white light as R:G:B, having a ratio of 1:1:1. “Color” indicates light that is not perceived as white because it stimulates cone cells in a ratio R.sub.1:G.sub.1:B.sub.1 that is not equal to 1:1:1. Each color R.sub.1:G.sub.1:B.sub.1 has a “complementary color,” which we designate as R.sub.C:G.sub.C:B.sub.C, such that white light is perceived when a color and its corresponding complementary color enter the eye simultaneously. This is because adding a color and its complementary color yields a ratio of 1:1:1, equal to the stimulations of the eye created by white light. In other words, the ratio of the stimulation to the eye provided by the combination of a color and its complementary color, expressed as R.sub.1+R.sub.C:G.sub.1+G.sub.C:B.sub.1+B.sub.C, is equal to 1:1:1.

As an example, light having a wavelength that is perceived as blue-violet stimulates cone cells receptive to that wavelength (blue-violet), and is represented in this model by a ratio of 0:0:1. Light having a wavelength that is perceived as yellow stimulates cone cells receptive to corresponding wavelengths (red and green), and is represented in this model by a ratio of 1:1:0. When these two colors are perceived simultaneously, the additive ratio of their components is 1:1:1. Accordingly, white light is perceived. Blue-violet and yellow are thus complementary colors of light.

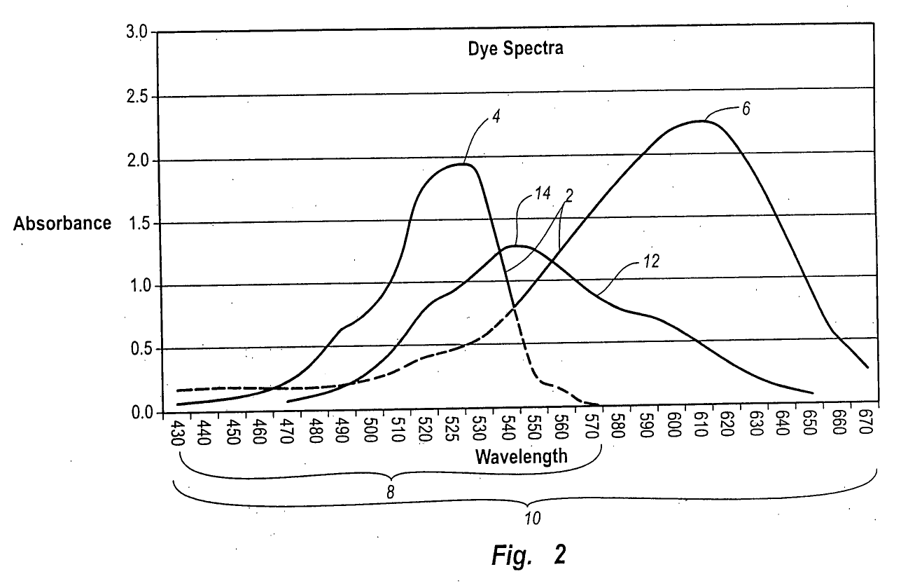

Conversely, a dye causes color perception by absorbing light having a particular wavelength. When a dye is illuminated by white light, one color is reflected (transmitted) by the dye; the complementary color of that color is absorbed by the dye. A spectral analysis of a dye can be used to illustrate, as in FIGS. 2 through 6, the wavelengths of light that the dye absorbs or transmits when illuminated by white light. The term “peak absorption wavelength” refers to that portion of the visible spectrum having a significantly higher level of absorption relative to its immediately adjacent portions.

Where all wavelengths of light corresponding to the visible spectrum are reflected from an object, the color of that object is perceived as white. Conversely, where all wavelengths of light corresponding to the visible spectrum are completely absorbed, the color of the object is perceived as black. When various wavelengths of light are absorbed differently, various colors are perceived.

If a white object is coated with a dye and then illuminated by white light, the light that is not absorbed by the dye is transmitted and perceived as color. The definition of a color thus perceived may be described either by referring to the wavelengths of the visible spectrum that the dye absorbed or by referring to the wavelengths of the visible spectrum that the dye transmitted. For the purposes of the present invention, all wavelengths used to numerically describe a dye are defined according to the wavelengths of the visible spectrum that the dye absorbs. For example, a dye that is perceived as red (e.g. it transmits wavelengths of the visible spectrum that the eye perceives as red), will be referred to herein as a dye that absorbs cyan-blue—the complimentary color of red when combining colors of light. A dye may also be described by those skilled in the art based upon the wavelengths of the visible spectrum that the dye transmits.

An exemplary non-invasive tooth-whitening composition suitable for home use is disclosed in U.S. Pat. No. 6,030,222 to Tarver (“Tarver”). Tarver teaches a whitening composition that includes a dye that, when absorbed by a tooth, causes the tooth to reflect a color of light that is whiter than the initial color of light reflected by the tooth. According to Tarver, the dye that most effectively causes this change in the light reflected by a tooth is a dye that reflects light having a wavelength that corresponds to a range from violet to blue-violet, this range of colors being a complementary color to the normal yellowish or off-white coloration of discolored teeth.

The effectiveness of the compositions disclosed in Tarver, however, is limited by the fact that the disclosed violet or blue-violet dye comprises unspecified quantities of red and blue dyes. The violet or blue-violet dye disclosed by Tarver has multiple peak absorption wavelengths, as illustrated at points 4 and 6 in FIG. 2. These multiple peak absorption wavelengths combine in the invention disclosed by Tarver to create a dye having a composite reflection or appearance in a range from violet to blue-violet. While theoretically a valid technique, such a combination of red and blue dyes is not as effective as the present invention. It appears that such combinations are subject to separation, either at a macroscopic or molecular level, once applied to teeth. Each component of the dye mixture may be absorbed differently by tooth surfaces, altering the light absorption characteristics of the initial dye mixture by leaving one of the peak absorption wavelengths more prominent in the dye after application to a tooth.

The compositions disclosed in Tarver present peripheral problems as well. The heterogeneous dye mixture taught by Tarver may cause discoloration of surrounding oral tissues because components of the dye mixture are preferentially absorbed by different oral tissues. When these dye mixtures are applied to teeth, the gums, tongue and surrounding oral tissues may display a red tint due to the continued presence of red dye within the initial dye mixture.

The present invention relies upon a single dye or combination of dyes having a dominant peak absorption wavelength corresponding to the complementary color of violet or blue-violet light, rather than, as Tarver discloses, a dye or combination of dyes having multiple peak absorption wavelengths that, taken together, are perceived as corresponding to the complementary color of violet or blue-violet. The whitening effect of a dye having a dominant peak absorption wavelength is significantly superior to a compound comprising multiple peak absorption wavelengths, as disclosed in Tarver. Further, a compound according to the present invention has been demonstrated to cause significantly less discoloration of adjacent oral tissues compared to the compound disclosed in Tarver.

Referring to FIG. 1, it is apparent that when an object is illuminated by white light, the wavelength of light corresponding to the color that is perceived—i.e. the light reflected from the object—is the complementary color of the wavelength of light corresponding to the color absorbed by the object. For example, a color perceived as blue-violet is caused by absorption of all yellow light (i.e., light having a wavelength between 560-595 nm). Likewise, a color perceived as yellow is caused by absorption of all blue-violet light (i.e. light having a wavelength between 400-480 nm). Yellow and blue-violet are complementary colors of light.

The present invention exploits the relationship between colors by disclosing a dye having a dominant peak absorption wavelength that is a complementary color to the absorption wavelengths typical of a yellowed tooth. When such a dye is added to the surface of the yellowed tooth, all of the colors of the visible spectrum are represented. In this manner, the visible wavelengths of light are substantially all present, resulting in a whiter appearance of the tooth.

A typical healthy tooth consists of an enamel surface, a dentin sub-layer beneath the enamel surface, and a central pulp chamber. The enamel surface comprises a highly calcified material that is preferably very hard and durable to allow chewing a wide variety of foods. The dentin sub-layer is also durable but considerably softer than the enamel. Dentin tends to have more of a yellowish hue compared to enamel, which tends to be white. Enamel may also be somewhat clear or translucent, which may enable yellowish tints of the dentin to show through the enamel. A tooth exhibiting a darker color than desired may also be the result of staining of the enamel, or even of the darker color of the pulp chamber being visible through the sometimes translucent enamel and dentin layers of the tooth.

Depending on the thickness, opacity and whiteness of the enamel, the color reflected by a person's teeth can vary widely between various shades of white, off-white, yellow, and yellow-green. The enamel surfaces of teeth can also accumulate plaque and tartar, which may increase their yellowish coloration.

In a preferred embodiment of the present invention, a small amount of dye is applied to a tooth, where that dye reflects a wavelength of light corresponding to violet or blue-violet. Because the violet or blue-violet reflected by the dye is a complementary color of the yellowish tints reflected by the tooth, the light reflected from the tooth with the dye has a whiter appearance than the tooth alone. In a preferred embodiment, a dye is used that has a dominant peak absorption wavelength within the range of 480 to 660 nm. Tarver, in the prior art, discloses the use of a dye or a combination of dyes that—though appearing violet or blue-violet—have multiple peak absorption wavelengths; using a dye with a dominant peak absorption wavelength can cause an otherwise yellowish tooth to appear substantially whiter than using a dye as disclosed in Tarver.

Referring now to FIG. 2, a combination of red and blue dyes that appear as violet may exhibit a spectral identity 2 having two peak absorption wavelengths, wherein a first peak 4 corresponds to a peak absorption wavelength unique to the red dye and a second peak 6 corresponds to a peak absorption wavelength unique to the blue dye. The resulting wavelength 2 is thus not truly violet, but simply the sum of the red wavelength 8 and the blue wavelength 10 as perceived by the eye. A dye according to the present invention exhibits a spectral identity 12 having a dominant peak absorption wavelength 14 corresponding to a point intermediate between the peak absorption wavelengths 4 and 6 for red and blue, respectively, and corresponding to the visible color of violet.

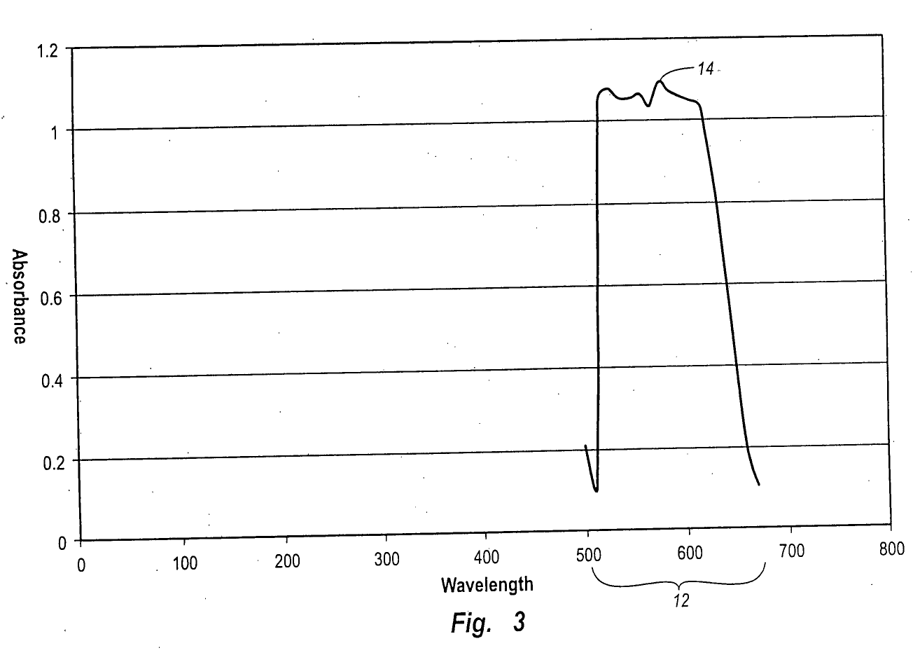

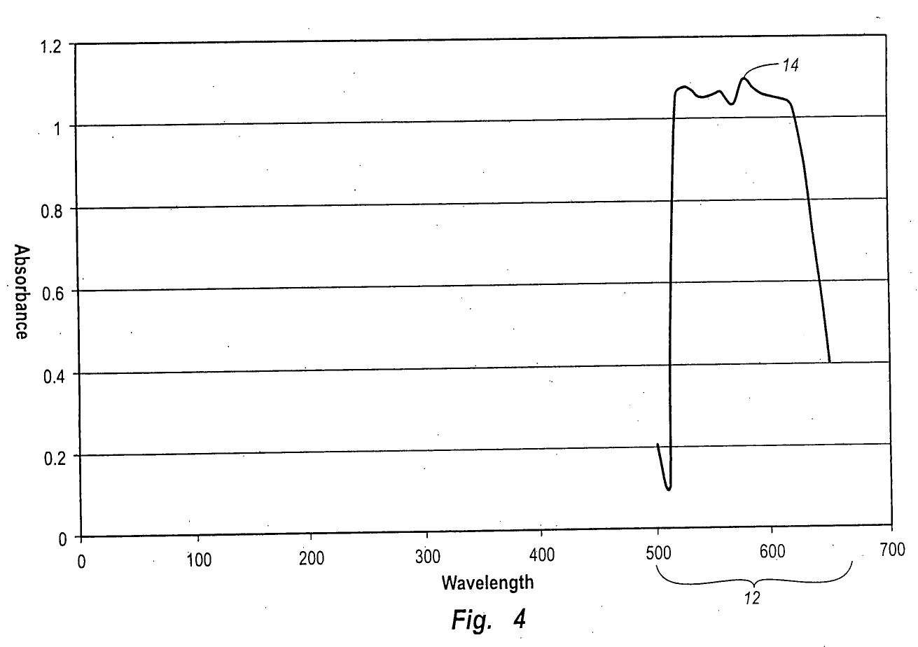

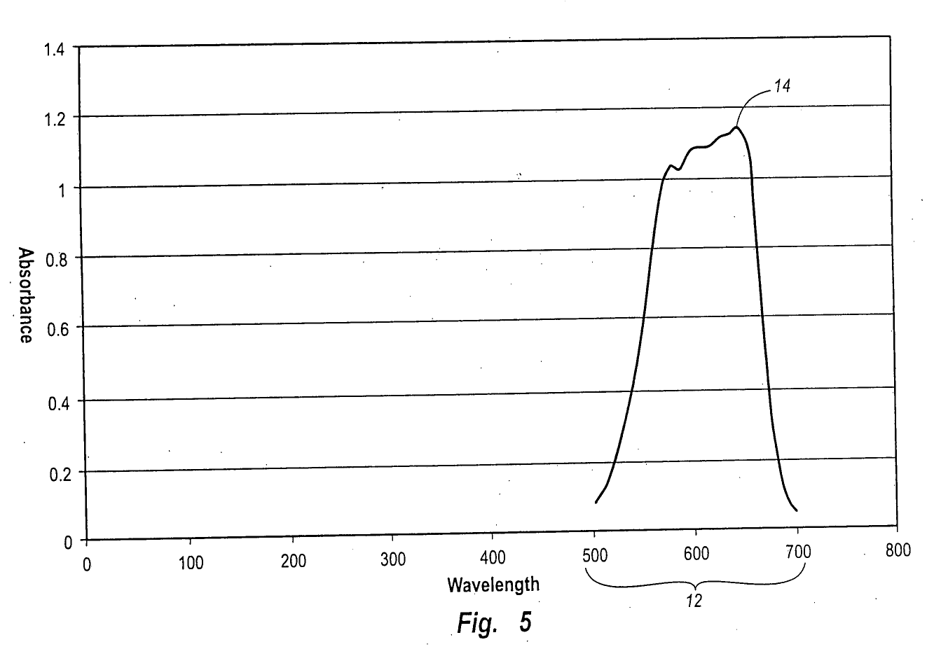

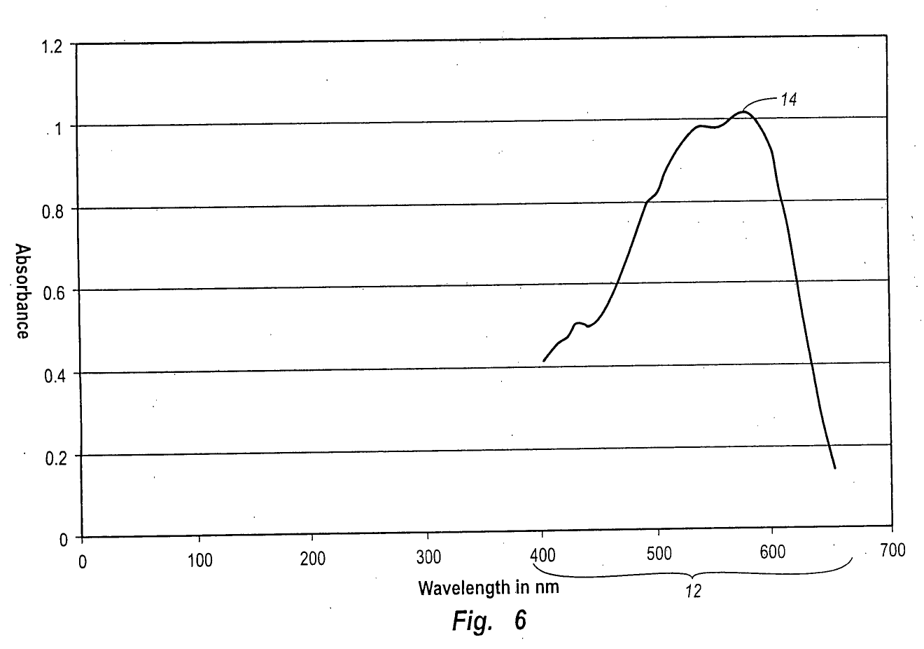

Referring now to FIGS. 3 through 6, a dye in accordance with a preferred embodiment of the present invention comprises a dye having a dominant peak absorption wavelength in the range of 480 to 660 nm, more preferably in a range from 500 to 620 .mu.m, and most preferably in a range from 540 to 595 nm. A dye may comprise, for example, FD&C Red No. 3, D&C Red No. 33, FD&C Red No. 40, FD&C Blue No. 1, FD&C Blue No. 2, D&C Violet No. 2, Acid Violet No. 17, Carmine, Blueberry Additive, or any other artificial or natural colorant known to those in the art that, when used singly or in combination, has a dominant peak absorption wavelength in a range from 480 to 660 nm. The attached figures illustrate the spectral analysis of several dyes used according to preferred embodiments of the present invention, with the peak absorption wavelengths of each being shown. FIG. 3 illustrates a spectral analysis of Acid Violet No. 17; FIG. 4 illustrates a spectral analysis of D&C Red No. 33; FIG. 5 illustrates a spectral analysis of FD&C Blue No. 1; and FIG. 6 illustrates a spectral analysis of Carmine.

The distinction between a dye having one dominant peak absorption wavelength and a dye having multiple peak absorption wavelengths is of paramount importance to the effectiveness of a tooth dye as taught by the present invention. Indeed, results far superior to those achieved by dyes as disclosed by Tarver have been achieved by applying a dye having one dominant peak absorption wavelength, as disclosed herein.

Moreover, a dye according to a preferred embodiment of the present invention avoids the negative effects seen in the prior art, in which the red component of a dye was seen to cause staining of oral tissues. Red staining may occur where any dye made according to the prior art retains a discrete chemical structure corresponding to red dye. FD&C Red No. 3 is particularly prone to have a red staining effect due to exposed iodine molecules bordering the chemical structure.

In one embodiment of the present invention, the disclosed tooth whitening composition further comprises a carrier. A preferred class of carriers within the scope of the present invention comprises mouthwash or another hydrophilic carrier designed to wash or be sprayed onto a person's teeth. Such carriers enable a dye used according to the present invention to be evenly and uniformly dispersed over and absorbed into a person's teeth. Moreover, hydrophilic carriers enable rapid absorption of a dye by a person's teeth because such carriers are attracted by the aqueous environment surrounding teeth. By way of example and not limitation, a mouthwash or other hydrophilic carrier may comprise any combination of the following: water, alcohols, glycerin and other polyhydric alcohols, polyols, ketones, aldehydes, carboxylic acids, carboxylic acid salts, or amines.

According to another embodiment of the present invention, a gelling agent may be used as a carrier to yield a gel or more viscous liquid for application to a person's teeth. Such possible gelling agents include, but are not limited to, polycarboxylic acids, polycarboxylic acid salts, polysaccharides, polysaccharide derivatives, proteins, protein derivatives, polyalkylene oxides, fumed silica, and the like.

According to another embodiment of the present invention, a dye may be used in combination with a toothpaste or other dentifrice used to clean, protect, or whiten a person's teeth. For example, the present invention may be used in combination with brush-on applications, prophylaxis pastes for professional cleaning applications, temporary plastic strips that are topically applied to teeth, sealants, applications involving curing lights, applications involving laser whitening treatments, carbomide peroxide gel whitening treatments, peroxide gel whitening treatments, or any other tooth whitening, protecting, or cleaning treatment known to those in the art.

According to yet another embodiment of the present invention, a dye may be incorporated into a chewing gum that is used as a carrier, such that the dye is released gradually as the gum is chewed. A gum used as a carrier for a dye according to the present invention may comprise, for example, sorbitol, gum base, mannitol, glycerin, acesulfame potassium, aspartame, and flavoring.

In yet another embodiment of the present invention, the tooth whitening composition comprises a carrier such as a food product, and the disclosed dye. Preferably, the carrier in this embodiment comprises a starch, cereal, beverage, candy or confectionery. Food products that maximize contact time between the food product and a person's tooth, such as taffy, hard candy, beverages, and other liquid or slowly degradable food products are especially preferred as a means to facilitate absorption of the dye by a person's tooth.

In still another embodiment of the present invention, two dyes that when used together have the whitening effect disclosed herein may be administered in separate stages, rather than in a single operation. In one non-limiting example of this embodiment, a blue dye is administered within a mouthwash, followed by a red dye that is administered within a dentifrice. The cumulative effect of these operations may then yield the results disclosed herein.

A tooth whitening composition in accordance with the present invention may be quickly and easily implemented by, for example, administering the disclosed tooth whitening composition to a person for ordinary consumption where the composition comprises a carrier such as a food product, or by selectively applying the tooth whitening composition directly to a person's tooth by way of an applicator stick, a toothbrush, an adhesive, or any other means known to those in the art.

### EXAMPLE 1

A beverage within the scope of the present invention was prepared by mixing with purified water the following two components: FD&C Blue No. 1 in a concentration of 0.04 mg/ml and FD&C Red No. 3 in a concentration of 0.033 mg/ml.

Test subjects drinking this water experienced an increase in tooth whiteness, as measured by an X-rite ShadeVision.™. tooth color measurement instrument.

### EXAMPLE 2

A food product within the scope of the present invention was prepared by mixing a carrier of 500 ml of Scope.RTM. brand mouthwash with a solution containing 3025 ul of blue solution and 3375 ul of red solution. The blue solution comprised FD&C Blue No. 1 in a concentration of 6.05 ul/ml, or 15 mg/ml in the final mixture. The red solution comprised FD&C Red No. 33 in a concentration of 6.75 ul/ml, or 15 mg/ml in the final mixture.

Test subjects used this mixture as a mouthwash and experienced an increase in tooth whiteness.

### EXAMPLE 3

A food product within the scope of the present invention was prepared by mixing a carrier of 50 g of Arm & Hammer.RTM. brand toothpaste with a solution containing 550 ul of red solution and 605 ul of blue solution. The blue solution comprised FD&C Blue No. 1 in a concentration of 12.10 ul/g, or 60 mg/ml in the final mixture. The red solution comprised FD&C Red No. 33 in a concentration of 11.00 ul/g, or 60 mg/ml in the final mixture.

Test subjects used this mixture as a dentifrice and experienced an increase in tooth whiteness.

### EXAMPLE 4

A food product within the scope of the present invention was prepared by adding Acid Violet No. 17 to a carrier of Scope.RTM. brand mouthwash in a concentration of 0.048 ml of dye to each ml of mouthwash.

Test subjects used this mixture as a mouthwash and experienced an increase in tooth whiteness.

As provided above, at least some embodiments of the present invention take place in association with applying one or more residual colors or dyes to the surface of a tooth in order to complete the color spectrum of the tooth surface or more closely complete the color spectrum of the tooth surface, thereby enhancing the appearance of the tooth since a complete color spectrum appears white.

In some embodiments, one or more residual colors are applied to the surface of the tooth to enhance the appearance thereof. In one embodiment, a residual color includes a shade of violet, wherein a red-violet reduces the yellow appearance of the tooth and a blue-violet enhances the brightness of the tooth.

In some embodiments, a two step process is utilized wherein a red-violet residual color is applied to the surface of a tooth in a first step to enhance the color of the tooth, causing the tooth to appear whiter. In a second step, a blue-violet residual color is applied to the surface of the tooth to enhance the brightness of the tooth, causing the tooth to appear brighter. In another embodiment, the blue-violet residual color is applied first and then the red-violet residual color is applied second. In yet another embodiment, the blue-violet residual color and the red-violet residual color are simultaneously applied.

In a further embodiment, the one or more residual colors are applied through utilization of a carrier. Examples of carriers include a gel (e.g., a glycerin or other type of gel), a toothpaste, a mouth wash, a liquid or beverage, a solid, a gas, a food, or any other type of carrier that is capable of applying a dye to the surface of a tooth.

In one embodiment, a bleaching step is performed to the surface of the tooth followed by the application of a residual color onto the surface of the tooth. In a further embodiment, the application of the residual color in combination with a step of bleaching the surface of the tooth effectively tones the tooth and thus enhances the appearance of the tooth. Further, application of the residual color prevents over bleaching the tooth surface.

In some embodiments, the residual color comprises a dual dye. In other embodiments, the residual color comprises a single dye.

In some embodiments, an intensity range is utilized based upon the current color of the tooth surface. Thus, for example, a more intense residual color is applied to a darker tooth surface color to enhance the appearance of the tooth. Accordingly, the intensity of the residual color is directly proportional to the darkness of the tooth prior to application of the residual color, whether the darkness of the tooth is due to natural colors and/or stains to the surface of the tooth. While embodiments embrace all types of intensities, some implementations embrace an intensity of the dye or residual color being no more than 1% of the total volume or weight of the product being applied to the tooth surface. In other embodiments, the intensity of the dye or residual color is no more than 5% of the total volume or weight of the product being applied to the tooth surface. In other embodiments, the intensity of the dye or residual color is no more than 10% of the total volume or weight of the product being applied to the tooth surface. In other embodiments, the intensity of the dye or residual color is more than 10% of the total volume or weight of the product being applied to the tooth surface.

In a further embodiment, a residual color includes a yellow shade applied to the surface of a tooth that appears gray. The application of a residual color that includes a yellow shade enhances the appearance of an otherwise gray tooth surface, by causing the tooth to appear whiter.

Thus, as discussed herein, embodiments of the present invention embrace enhancing the appearance of teeth. In particular, embodiments of the present invention relate to effective, non-invasive and non-destructive systems and methods that provide an appearance of increased whiteness and/or brightness of the teeth, thereby enhancing the appearance of the teeth.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes that come within the meaning and range of equivalency of the claims are to be embraced within their scope.

What is claimed is:

1. A method for enhancing the appearance of a tooth, the method comprising:
applying a residual color to a surface of the tooth to complete a color spectrum of the tooth, wherein the completed color spectrum causes the tooth to appear whiter.

2. A method as recited in claim 1, wherein the residual color comprises violet.

3. A method as recited in claim 2, wherein the violet is a red-violet.

4. A method as recited in claim 1, wherein the residual color comprises a red-violet and a blue-violet.

5. A method as recited in claim 4, wherein the red-violet causes the tooth to appear whiter and the blue-violet causes the tooth to appear brighter.

6. A method as recited in claim 1, wherein the surface of the tooth appears gray prior to said application of the residual color, and wherein the residual color comprises yellow to cause the tooth to appear whiter.

7. A method as recited in claim 1, wherein said application comprises:
application of a first color to the surface of the tooth; and
application of a second color to the surface of the tooth.

8. A method as recited in claim 7, wherein said first color comprises a red-violet and said second color comprises a blue-violet.

9. A method as recited in claim 7, wherein said first color comprises a yellow and said second color comprises a blue-violet.

10. A method as recited in claim 1, wherein said application comprises:
bleaching the surface of the tooth; and
applying the residual color to the surface of the tooth.

11. A method as recited in claim 10, wherein said application of the residual color to the surface of the tooth comprises:
application of a first color to the surface of the tooth; and
application of a second color to the surface of the tooth.

12. A method as recited in claim 11, wherein said first color comprises a red-violet and said second color comprises a blue-violet.

13. A method as recited in claim 11, wherein said first color comprises a yellow and said second color comprises a blue-violet.

14. A method as recited in claim 10, wherein said applying the residual color to the surface of the tooth comprises applying the residual color through use of a carrier.

15. A method as recited in claim 14, wherein a dye that comprises the residual color is not more than 1% of the total amount of the carrier.

16. A method as recited in claim 14, wherein a dye that comprises the residual color is not more than 5% of the total amount of the carrier.

17. A method as recited in claim 14, wherein a dye that comprises the residual color is not more than 10% of the total amount of the carrier.

18. A method as recited in claim 1, wherein an intensity of the residual color corresponds to the darkness of the tooth surface prior to application of the residual color.

19. A tooth enhancing product comprising:
a red-violet residual color component for application onto an outside surface of a tooth to complete a color spectrum of the tooth and cause the tooth to appear whiter.

20. A tooth enhancing product as recited in claim 19, further comprising a blue-violet residual color component for application onto the outside surface of a tooth to cause the tooth to appear brighter.

21. A tooth enhancing product comprising:
a yellow residual color component for application onto an outside surface of a tooth to complete a color spectrum of the tooth and cause the tooth to appear whiter.

22. A tooth enhancing product as recited in claim 21, further comprising a blue-violet residual color component for application onto the outside surface of a tooth to cause the tooth to appear brighter.
